(19) United States
(12) Patent Application Publication
(10) Pub. No.: US 2014/0296693 A1
(43) Pub. Date: Oct. 2, 2014

(54) PRODUCTS OF MANUFACTURE AND METHODS USING OPTICAL COHERENCE TOMOGRAPHY TO DETECT SEIZURES, PRE-SEIZURE STATES AND CEREBRAL EDEMAS

(71) Applicant: THE REGENTS OF THE UNIVERSITY OF CALIFORNIA, Oakland, CA (US)

(72) Inventors: Devin K. BINDER, Fullerton, CA (US); Hyle PARK, Riverside, CA (US); Mike S. HSU, Chino, CA (US)

(73) Assignee: THE REGENTS OF THE UNIVERSITY OF CALIFORNIA, Oakland, CA (US)

(21) Appl. No.: 13/855,491

(22) Filed: Apr. 2, 2013

(51) Int. Cl. A61B 5/00

(52) U.S. Cl. CPC A61B 5/4094; A61B 5/0073
USPC 600/408; 600/425; 600/427

(57) ABSTRACT

In alternative embodiments, the invention provides compositions, products of manufacture and medical devices, and methods, using optical coherence tomography to monitor for a physiological event and/or a state prior to the physiological event. The invention also provides computer program products and computer implemented methods to capture, analyze, and display optical coherence tomography images of neural tissue to measure, detect and/or monitor for a physiological event and/or a state prior to the physiological event.

### GOVERNMENT RIGHTS

This invention was made with government support under grant numbers EB007241; NS059674; 5K08NS059674-05, and 5R00EB007241-05, awarded by the National Institutes of Health (NIH). The government has certain rights in the invention.

### TECHNICAL FIELD

This invention generally relates to medical devices, such as devices for optical coherence tomography, and medical diagnostics. In alternative embodiments, the invention provides compositions, products of manufacture and medical devices, and methods, using optical coherence tomography to measure, detect and/or monitor for a physiological event and/or a state prior to the physiological event. The invention also provides computer program products and computer implemented methods to capture, analyze, and display optical coherence tomography images of neural tissue to monitor for a physiological event and/or a state prior to the physiological event.

### BACKGROUND

Seizure activity causes neurons and glial cells to undergo changes which alter the way light propagates through brain tissue. Currently seizures are detected from deep brain structures with the use of electroencephalography (EEG). EEG recordings are made through electrodes placed on the scalp, on the brain surface, or inserted deep in the brain. EEG works through amplification of minute voltage potentials generated in brain tissue. As such it has inherently low signal to noise ratio and requires controlled conditions for optimal signal detection. Currently, attempts are underway to improve the early detection sensitivity of EEG through complex mathematical manipulations of EEG data, though at the current state of the art it is not possible to reliably predict seizures through real-time analysis of EEG signals.

There is currently no way to detect seizures optically in deep brain structures. Previous optical techniques have been limited to absorption, related changes, and detection at the cortical surface. Detection of deep brain seizure foci is restricted to EEG detection.

### SUMMARY

In alternative embodiments, the invention provides compositions, products of manufacture and medical devices, and methods, using optical coherence tomography to measure, detect and/or monitor for a physiological event and/or a state prior to the physiological event such as a seizure, or any event related to edema in the brain.

In alternative embodiments, the invention provides methods for using optical coherence tomography to monitor for a physiological event and/or a state prior to the physiological event in an individual, comprising:

(a) scanning a neural tissue with an optical coherence tomography (OCT) at a predetermined location, and generating an optical coherence signal;

(b) analyzing characteristics of the optical coherence signal on the neural tissue as a function of time; and

(c) determining OCT signal changes before the physiological event in the neural tissue becomes clinically manifested;

wherein optionally the method comprises determining a signature signal that comprises changes, or a repeatable pattern, in signal intensity specifically associated with a seizure or a pre-seizure activity,

wherein optionally the method is a computer implemented method comprising use of a computer program product to capture, analyze, and display optical coherence tomography images of the neural tissue to monitor for a physiological event and/or a state prior to the physiological event.

In alternative embodiments, the physiological event is a seizure and the method further comprises mediating or manipulating the neural activity of the neural tissue before onset of the seizure upon a determination of a signature temporal pattern.

In alternative embodiments, the mediating of the neural activity of the neural tissue comprises a mediation sufficient to prevent a seizure. In alternative embodiments, the mediating of the neural activity of the neural tissue comprises a mediation sufficient to reduce the time (duration) of and/or the severity of seizure.

In alternative embodiments, determining the optical intensity of the optical coherence signal comprises determining a threshold value of the optical intensity. In alternative embodiments, determining a threshold value of the optical intensity comprises determining a threshold value during one or more time windows of decreasing optical intensity. In alternative embodiments, determining a signature signal of the optical intensity of the optical coherent signal comprises determining a threshold value of the time derivative of the optical intensity.

In alternative embodiments, determining a threshold value of the time derivative of the optical intensity comprises determining a threshold value of the time derivative of the optical intensity during one or more time windows of decreasing optical intensity.

In alternative embodiments, the invention provides methods for using optical parameters in detection of a seizure and/or a pre-seizure state in an individual, comprising:

(a) scanning a neural tissue with optical coherence tomography;

(b) detecting a magnitude of optical intensity reflected by the neural tissue as a function of time; and

(c) detecting changes in neural architecture that result from water and ion migration preceding and during specific brain electrical activity,

wherein detecting changes in the neural architecture detects a seizure and/or a pre-seizure state, and optionally the method comprises determining a signature signal that comprises changes, or a repeatable pattern, in optical intensity specifically associated with a seizure or a pre-seizure activity, and optionally the method is a computer implemented method comprising use of a computer program product to capture, analyze, and display optical coherence tomography images of the neural tissue.

In alternative embodiments, the invention provides methods for using optical parameters in detection of seizure and pre-seizure states comprising:

(a) scanning a neural tissue with an optical coherence tomography;

(b) detecting a magnitude of optical intensity reflected by neural tissue as a function of time; and

(c) detecting changes in cell volume and detecting changes in the extracellular space which decrease optical scattering through affected neural tissue,

wherein detecting changes in the cell volume and extracellular space which decrease optical scattering detects a seizure and/or a pre-seizure state, and optionally the method comprises determining a signature signal that comprises changes, or a repeatable pattern, in optical intensity specifically associated with a seizure or a pre-seizure activity, and optionally the method is a computer implemented method comprising use of a computer program product to capture, analyze, and display optical coherence tomography images of the cell or neural or extracellular tissue.

In alternative embodiments, the optical coherence tomography is supplied through a fiber optic endoscope. In alternative embodiments, the optical coherence tomography is supplied through a fiber optic endoscope, and the method comprises implanting an endoscope into a seizure foci of an individuals with epilepsy, wherein optionally the detecting of a pre-seizure state acts as an early warning device.

In alternative embodiments, the method further comprises utilizing variations of optical coherence tomography on the neural tissue with either broadband or specific wavelengths of radiation in the visible, near-infrared, and/or infrared region.

In alternative embodiments, the method comprises determining a signature signal that comprises changes, or a repeatable pattern, in signal intensity associated with a seizure or a pre-seizure activity.

In alternative embodiments, the method comprises determining a signature signal that comprises changes, or a repeatable pattern, in signal or optical intensity associated with pathologic brain swelling before measurable late sequelae of increased intracranial pressure or hemodynamic changes,

wherein optionally the method, by detecting signal or optical intensity associated with pathologic brain swelling before measurable late sequelae of increased intracranial pressure or hemodynamic changes, acts as an early warning of increased intracranial pressure or hemodynamic changes.

In alternative embodiments, the method further comprises using an optical fiber edema probe incorporated into an intracranial monitoring device or inserted as a standalone probe into an area of interest in the neural tissue, and analyzing optical scattering and absorption related changes to provide an edema index on a continuous basis.

In alternative embodiments, determining the OCT signal changes comprises use of light polarization information, the optical phase of the signal, spectroscopic information, and changes thereof.

In alternative embodiments, the invention provides products of manufacture or devices, such as a medical device, comprising an optical coherence tomography capable of monitoring for a physiological event and/or a state prior to the physiological event in an individual, comprising: (a) scanning a neural tissue with an optical coherence tomography (OCT) at a predetermined location, and generating an optical coherence signal; (b) analyzing characteristics of the optical coherence signal on the neural tissue as a function of time; and (c) determining OCT signal changes before the physiological event in the neural tissue becomes clinically manifested;

wherein optionally the product of manufacture or a device can determine a signature signal that comprises changes, or a repeatable pattern, in signal intensity specifically associated with a seizure or a pre-seizure activity,

wherein optionally the product of manufacture or device further comprises a computer program product for enabling a computer implemented method to capture, analyze, and display optical coherence tomography images of the neural tissue to monitor for a physiological event and/or a state prior to the physiological event.

In alternative embodiments, the products of manufacture or devices of the invention further comprise an artificial neural-network (ANN) based computer chip or a computer program product to enhance the specificity of an imaging system.

In alternative embodiments, the products of manufacture or devices of the invention further comprise a computer program product having a computer implemented method or a software programmed for screening for a “yes or no” response; wherein optionally for a yes (optionally, yes for seizure or edema)) or a no (optionally, no for seizure or edema) basis, and optionally comprising a memory, and a relaying or a telecommunication or an internet relay, and/or a display for a user component to store, transmit and/or display the “yes or no” response.

In alternative embodiments, the products of manufacture or devices of the invention further comprise a sensor, wherein the sensor can optionally detect and/or record radiation exposure specific or cumulative for an operator and/or for subject safety.

In alternative embodiments, the invention provides computer-implemented method for, or capable of, implementing a method of the invention. In alternative embodiments, the invention provides computer program products for processing data acquired in implementing a method of the invention, or the computer-implemented method of the invention. In alternative embodiments, the invention provides storage devices storing program instructions executable by a processor to run, process and/or implement: (a) a computer-implemented method of the invention; or (b) a computer program product of the invention. In alternative embodiments, the invention provides computer-implemented systems for providing an application access to an external data source or an external server process via a connection server, and providing the ability to store values associated with the plurality of data points and/or the plurality of data elements, and an application for running, processing and/or implementing: (a) a computer-implemented method of the invention; or (b) a computer program product of the invention. In alternative embodiments, the invention provides systems, comprising: a memory configured to: store values associated with a plurality of data points and/or a plurality of data elements, and a processor adapted to execute program code stored in the memory to: run, process and/or implement: (a) a computer-implemented method of the invention; or (b) a computer program product of the invention.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1 schematically illustrates exemplary product of manufacture or device of the invention.

FIG. 2 illustrates the early detection of cerebral edema with a single-wavelength NIR laser diode-power meter system: FIG. 2A, illustrates an image from a dual fiberoptic probe implanted into the right cerebral cortex through a burr hole while the ICP monitor is placed contralaterally; FIG. 2B, upper graph, graphically illustrates intracranial pressure recordings before and after the injection of distilled water (30% body weight, i.p.; black arrows); FIG. 2B, lower graph, graphically illustrates reflectance measurements in nanowatts (nW) obtained with dual fiberoptic probe; FIG. 2B, Left inset: upper graph, graphically illustrates baseline ICP waveform with low (<5 mm Hg) variability; FIG. 2B, Right inset: upper graph, graphically illustrates ICP waveform at higher pressure with increased amplitude of variability (>5 mm Hg) compared to lower ICP; FIG. 2C, graphically illustrates time interval between water injection (time point 0), optical trigger and defined threshold ICP values; as discussed in further detail, below.

FIG. 3A illustrates brain images from a water intoxication model of cerebral edema that demonstrated that the OCT imaging modality of this invention is capable of detecting and monitoring cerebral edema through morphological and light reflectivity changes with greater spatial and temporal resolution than the NIRS fiberoptic system; and FIG. 3B graphically illustrates these results; as discussed in further detail, below.

FIG. 4 illustrates an exemplary OCT system of the invention with fiberoptic probes: FIG. 4(A) is an exemplary SLD source; FIG. 4 (B) is an exemplary keyboard and touch screen monitor; FIG. 4(C) is an exemplary reference Arm; FIG. 4(D) is an exemplary spectrometer; FIG. 4(E) is an exemplary computer; and, FIG. 4(F) is an exemplary fiberoptic endoscope sample arm.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the invention provides products of manufacture and medical devices, and methods for making and using them, comprising use of optical coherence tomography to measure, detect and/or monitor for a physiological event and/or a state prior to the physiological event, for example, seizures or cerebral edema. The invention also provides computer program products and computer implemented methods to capture, analyze, and display optical coherence tomography images of neural tissue to monitor for a physiological event (e.g., seizures or cerebral edema) and/or a state prior to the physiological event.

Our data indicate that scattering changes are associated with cellular changes which begin before a seizure occurs, e.g., to detect and predict neuronal activity and seizures in the brain, e.g., in the cerebral cortex. Thus, this invention is not limited to detecting only changes in diffuse reflectance which primarily arise from blood perfusion changes. In one embodiment, because optical changes occur before seizures manifest on EEG, the compositions and methods of the invention, using an optical paradigm, are ideally suited to act as an early warning detector of a seizure. Thus, in alternative embodiments, the compositions and methods of the invention, by detecting and/or predicting neuronal activity and by early detection of seizures, can prevent or decrease the significant secondary injury to the brain due to cerebral edema.

We have developed compositions, e.g., products of manufacture, and methods, for detecting cerebral edema directly through the use of optics [1]. We have discovered that the scattering of near-infrared (NIR) light is a sensitive and direct indicator of early cerebral edema [1]. In alternative embodiments, to achieve maximum temporal and spatial resolution, products of manufacture and methods of the invention use optical coherence tomography (OCT), an “optical ultrasound” imaging modality. In alternative embodiments, the OCT also uses real-time online analysis of optical tissue changes. In alternative embodiments, compositions, e.g., products of manufacture, and methods of the invention comprise an automated algorithm for detection of cerebral edema using OCT imaging. In alternative embodiments, compositions, e.g., products of manufacture, and methods of the invention are minimally-invasive, e.g., configures as a head-mounted fiberoptic OCT monitoring system for e.g., detection of seizures and/or cerebral edema.

In alternative embodiments, compositions, e.g., products of manufacture, and methods of the invention comprise use of optical coherence tomography (OCT) as an advanced neuromonitoring system to detect and monitor changes in brain tissue resulting from cerebral edema. In alternative embodiments, a thin fiberoptic probe is placed on the surface of the brain and connected to an OCT system. Changes in optical properties of brain tissue can be continuously monitored, and resulting data are acquired and interpreted by analytical software, and an online detection algorithm can be used to warn the caregiver of impending edema. This will provide caregivers (e.g., physicians and nurses) accurate information at earlier time points than standard intracranial pressure (ICP) monitoring and thus allow early point-of-care clinical intervention prior to progression of edema and clinical deterioration with brain herniation. In alternative embodiments, a complete system includes (comprises) the fiberoptic probes, OCT and/or MRI system, data acquisition/storage computer station; and also can comprise a complete analytical software package which interprets the acquired data to offer an integrated advanced neuromonitoring solution for early cerebral edema detection.

In alternative embodiments, compositions, e.g., products of manufacture, and methods of the invention comprise or comprise use of a monitoring system that utilizes thin fiberoptic probes which can be implanted, e.g., through FDA-approved cranial (Camino) bolt device (e.g., marketed by Integra) for brain surface access and skull fixation, e.g., in neuro ICU patients.

In alternative embodiments, compositions, e.g., products of manufacture, and methods of the invention comprise or comprise use of OCT to detect changes in tissue optical properties, e.g., changes in tissue optical properties associated with a cerebral edema or related event. OCT can be thought of as an optical analog of ultrasound imaging, in which the intensity and time delay of reflected light is used to generate cross-sectional images of tissue microstructure with micron-level resolution [10]. In alternative embodiments, compositions, e.g., products of manufacture, and methods of the invention comprise or comprise use second generation instruments that utilize Fourier-domain, rather than time domain, detection to realize several orders of magnitude in system sensitivity, and that can be used to increase acquisition speed and stability [11-14]. In alternative embodiments, compositions, e.g., products of manufacture, and methods of the invention comprise or comprise use optical scattering and reflectivity coefficients, which can be important optical parameters to monitor during cerebral edema, and can be extracted from OCT images accurately and with more than sufficient spatiotemporal resolution.

In alternative embodiments, the invention provides detection and monitoring systems comprising optical coherence tomography (OCT) and computer program products to obtain, store, analyze, display and transmit information about edema and/or brain tissue swelling. In alternative embodiments, the compositions and methods comprise use of a fiber optic probe that is implanted into a hole on the patient's skull; this allows for penetration of the OCT signal into the brain. In alternative embodiments, OCT images of the brain are obtained and data is interpreted, stored and/or transmitted by computer program products (e.g., software) to detect changes in the brain due to swelling or edema. In alternative embodiments, compositions, e.g., products of manufacture, and methods of the invention, have improved sensitivity temporally and spatially, as well as minimal invasiveness, when applied to detecting cerebral edema.

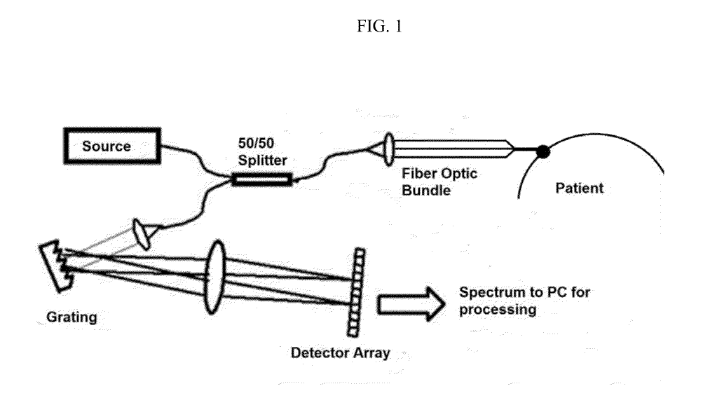

In alternative embodiments, compositions, e.g., products of manufacture, and methods of the invention comprise or comprise use optical coherence tomography (OCT) imaging system to detect and monitor changes in brain tissue resulting from a cerebral edema or a related cerebral event. In alternative embodiments, a thin fiberoptic probe is placed on through or on the surface of the cranium and connected to the OCT system. In alternative embodiments, changes in optical properties of brain tissue are continuously monitored, resulting data are acquired and interpreted by analytical software, and an online detection algorithm is used to warn the attending physician or nurse of impending edema. In alternative embodiments, systems of the invention are intended for usage and installation by trained technicians, physicians and nurses. In alternative embodiments, once installed, a system of the invention can provide caregivers (e.g., physicians and nurses) accurate information about edema and brain swelling at earlier time points than standard intracranial pressure (ICP) monitoring, and thus can allow early point-of-care clinical intervention prior to progression of edema and clinical deterioration with brain herniation. In alternative embodiments, systems of the invention comprise fiberoptic probes, optical imaging system, data acquisition/storage computer station and/or an analytical software package to collate, store and interpret the acquired data to offer an integrated advanced neuromonitoring solution for early cerebral edema detection. An exemplary product of manufacture or device of the invention is schematically illustrated in FIG. 1.

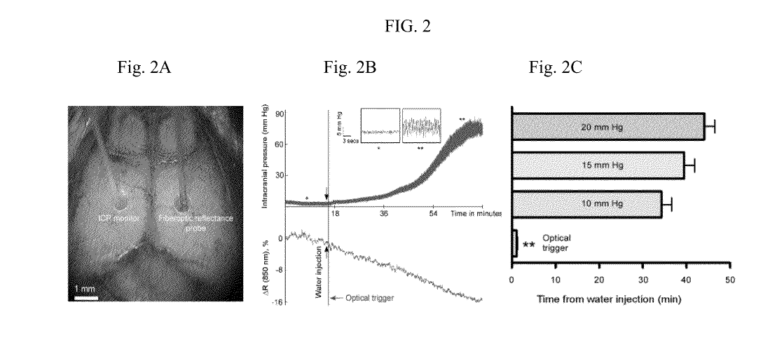

FIG. 2 illustrates the early detection of cerebral edema with a single-wavelength NIR laser diode-power meter system: FIG. 2A, Left: dual fiberoptic probe is implanted into the right cerebral cortex through a burr hole while the ICP monitor is placed contralaterally; FIG. 2B, Middle top: intracranial pressure recordings before and after the injection of distilled water (30% body weight, i.p.; black arrows); FIG. 2B, Middle bottom: reflectance measurements in nanowatts (nW) obtained with dual fiberoptic probe. In this example, the “optical trigger” (vertical blue line) occurs 31 minutes prior to reaching the pathologically increased ICP of 20 mm Hg. FIG. 2B, Left inset: baseline ICP waveform with low (<5 mm Hg) variability. FIG. 2B, Right inset: ICP waveform at higher pressure with increased amplitude of variability (>5 mm Hg) compared to lower ICP. FIG. 2C, Right: time interval between water injection (time point 0), optical trigger and defined threshold ICP values (n=3, mean±SEM). Optical trigger is detected significantly earlier than any of the ICP thresholds (p<0.001). See reference [1], below.

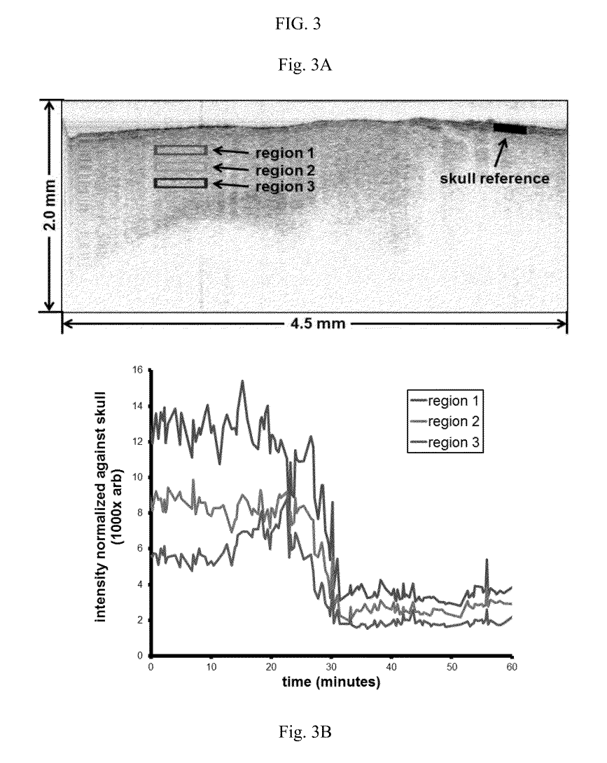

In alternative embodiments, OCT instruments of the invention can probe the subsurface structure of the brain, e.g., using animal models of cerebral edema, to determine the changes of various optical parameters from OCT images. Anesthetized animals are subjected to cerebral edema (via traumatic brain injury by controlled cortical impact device), and scanned with different OCT modalities through a thinned skull window immediately adjacent to the injury site. Simultaneously, intracranial pressure records (ICP) changes to compare its performance with that of imaging systems of this invention. We expect to observe changes in tissue reflectivity with the onset of cerebral edema. Indeed, our recent studies utilizing the water intoxication model of cerebral edema have demonstrated that the OCT imaging modality of this invention is capable of detecting and monitoring cerebral edema through morphological and light reflectivity changes with greater spatial and temporal resolution than the NIRS fiberoptic system described above, see FIG. 3.

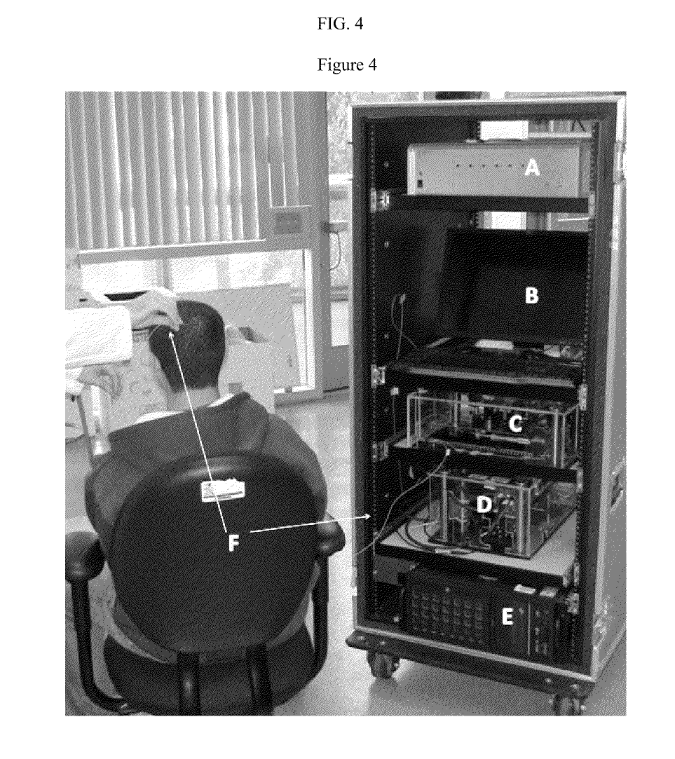

FIG. 4 illustrates an exemplary OCT system of the invention with fiberoptic probes. LEFT: A) SLD source. B) Keyboard and touch screen monitor. C) Reference Arm. D) Spectrometer. E) Computer. F) Fiberoptic endoscope sample arm. TOP: Tip of the fiberoptic endoscope. In alternative embodiments, light is passed from the source into the fiberoptic bundle, which contains both sample and reference arm probes. In alternative embodiments, in an exemplary finished system, the fiberoptic sample arm will be secured to the patient by the means of a cranial bolt. In alternative embodiments back-scattered light is received from the brain and passes into the detection arm. In alternative embodiments the detection arm light is processed in real time by the edema detection algorithm and a trigger occurs when alteration in back-scattered light indicates early cerebral edema.

In alternative embodiments, compositions, e.g., products of manufacture, and methods of the invention are minimally-invasive, head-mounted fiberoptic OCT monitoring systems for detection of e.g., cerebral edema. In alternative embodiments, compositions, e.g., products of manufacture, and methods of the invention comprise use of a head-mounted OCT system with minimally-invasive thin fiberoptic probes with greater spatial and temporal resolution than existing methods. In alternative embodiments, compositions, e.g., products of manufacture, and methods of the invention comprise use of a source (e.g., a superluminescent diode) to provide a light, which can be directed into fiberoptic probes and into the target of interest. In alternative embodiments, data from back-reflected light will be recorded and an image can be created from the data. In alternative embodiments, OCT platform data is integrated with the real-time detection algorithms to allow recognition of relevant changes in optical properties of edematous tissue and to automate the process of early edema detection. In alternative embodiments, with this software-hardware integration, the products of manufacture and methods of the invention are a point-of-care device optimized to provide accurate information to the bedside caregiver in real time.

We have demonstrated that multi-functional versions of OCT systems can be implemented using the unrestricted use of fiber-optic components [17], and can be adapted for endoscopic application [18, 19]. In alternative embodiments, compositions, e.g., products of manufacture, and methods of the invention comprise a head-mounted OCT driven platform utilizing fiberoptic probes to yield a minimally-invasive real-time system for detection of cerebral edema. The platform can be integrated with an automated detection software package. In alternative embodiments, compositions, e.g., products of manufacture, and methods of the invention are used for the detection of cerebral edema in the traumatic brain injury (TBI). In alternative embodiments, compositions, e.g., products of manufacture, and methods of the invention provide a superior alternative to ICP monitoring in detection of cerebral edema by offering In alternative embodiments, compositions, e.g., products of manufacture, and methods of the invention implement a polarization-sensitive OCT. In alternative embodiments, the application of a head-mounted endoscopic sample arm minimizes motion artifacts. In alternative embodiments, various configurations of the tip of the fiberoptic sample arm are used to optimize field of view for best tissue sampling.

### REFERENCES

1. Gill, A. S., et al., Early optical detection of cerebral edema in vivo. J Neurosurg, 2011. 114: p. 470-477.
2. Marmarou, A., Pathophysiology of traumatic brain edema: current concepts. Acta Neurochir Suppl, 2003. 86: p. 7-10.
3. Park, E., J. D. Bell, and A. J. Baker, Traumatic brain injury: can the consequences be stopped? CMAJ, 2008. 178(9): p. 1163-70.
4. Czosnyka, M. and J. D. Pickard, Monitoring and interpretation of intracranial pressure. J Neurol Neurosurg Psychiatry, 2004. 75(6): p. 813-21.
5. Attas, M., et al., Visualization of cutaneous hemoglobin oxygenation and skin hydration using near-infrared spectroscopic imaging. Skin Res Technol, 2001. 7(4): p. 238-45.
6. Nakajima, R., et al., Novel method for quantification of brain cell swelling in rat hippocampal slices. J Neurosci Res, 2004. 76(5): p. 723-33.
7. Piao, D. Q. and B. W. Pogue, Rapid near-infrared diffuse tomography for hemodynamic imaging using a low-coherence wideband light source. Journal of Biomedical Optics, 2007. 12(1): p. -.
8. Rasmussen, P., et al., Capillary-oxygenation-level-dependent near-infrared spectrometry in frontal lobe of humans. J Cereb Blood Flow Metab, 2007. 27(5): p. 1082-93.
9. Thiagarajah, J. R., M. C. Papadopoulos, and A. S. Verkman, Noninvasive early detection of brain edema in mice by near-infrared light scattering. J Neurosci Res, 2005. 80(2): p. 293-9.
10. Huang, D., et al., Optical coherence tomography. Science, 1991. 254(5035): p. 1178-81.
11. Mitsui, T., Dynamic range of optical reflectometry with spectral interferometry. Japanese Journal of Applied Physics Part 1-Regular Papers Short Notes & Review Papers, 1999. 38(10): p. 6133-6137.
12. Leitgeb, R., C. K. Hitzenberger, and A. F. Fercher, Performance of fourier domain vs. time domain optical coherence tomography. Optics Express, 2003. 11(8): p. 889-894.
13. de Boer, J. F., et al., Improved signal-to-noise ratio in spectral-domain compared with time-domain optical coherence tomography. Optics Letters, 2003. 28(21): p. 2067-2069.
14. Choma, M. A., et al., Sensitivity advantage of swept source and Fourier domain optical coherence tomography. Optics Express, 2003. 11(18): p. 2183-2189.
15. Schmitt, J. M., A. Knuttel, and R. F. Bonner, Measurement of Optical-Properties of Biological Tissues by Low-Coherence Reflectometry. Applied Optics, 1993. 32(30): p. 6032-6042.
16. Knuttel, A. and M. Boehlau-Godau, Spatially confined and temporally resolved refractive index and scattering evaluation in human skin performed with optical coherence tomography. Journal of Biomedical Optics, 2000. 5(1): p. 83-92.
17. Park, B. H., et al., Real-time fiber-based multi-functional spectral-domain optical coherence tomography at 1.3 mu m. Optics Express, 2005. 13(11): p. 3931-3944.
18. Pierce, M. C., et al., Effects of sample arm motion in endoscopic polarization-sensitive optical coherence tomography. Optics Express, 2005. 13(15): p. 5739-5749.
19. Kim, K. H., et al., Two-axis magnetically-driven MEMS scanning catheter for endoscopic high-speed optical coherence tomography. Optics Express, 2007. 15(26): p. 18130-18140.

### Optical Coherence Tomography (OCT)

In alternative embodiments, compositions and methods of the invention comprise or comprise use of Optical coherence tomography (OCT), an optical signal acquisition and processing method. OCT can with micrometer-resolution achieve three-dimensional images from within optical scattering media, e.g., cerebral tissue. Optical coherence tomography is an interferometric technique that can employ a near-infrared light. The use of relatively long wavelength light allows it to penetrate into the scattering medium, e.g., a brain. Any OCT device or methods of using OCT devices, or variations thereof, can be used, e.g., as described in U.S. Pat. No. 5,491,524; 5,795,295; 6,922,583; 7,610,074; 7,751,057; 7,983,737 or 7,999,945; or U.S. Pat. App. Pub. Nos. 20090185166; or 20090221920.

### Computer Systems and Data Storage Devices

In alternative embodiments, products of manufacture or devices of the invention comprise an optical coherence tomography capable of monitoring for a physiological event and/or a state prior to the physiological event in an individual, wherein optionally the product of manufacture or device further comprises a computer program product for enabling a computer implemented method to capture, analyze, and display optical coherence tomography images of the neural tissue to monitor for a physiological event and/or a state prior to the physiological event. In alternative embodiments, products of manufacture or devices or methods of the invention, in whole or in part, can require implementation using a machine, computer system or equivalent, within which a set of instructions for causing the computer or machine to perform any one or more of the protocols or methodologies of the invention may be executed. In alternative embodiments, the machine may be connected (e.g., networked) to other machines, e.g., in a Local Area Network (LAN), an intranet, an extranet, or the Internet, or any equivalents thereof. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. The term “machine” shall also be taken to include any collection of machines, computers or products of manufacture that individually or jointly execute a set (or multiple sets) of instructions to perform any one or more of the methodologies of the invention.

In alternative embodiments, an exemplary computer system of the invention, or system used to practice a method of the invention, comprises a processing device (processor), a main memory (e.g., read-only memory (ROM), flash memory, dynamic random access memory (DRAM) such as synchronous DRAM (SDRAM) or Rambus DRAM (RDRAM), etc.), a static memory (e.g., flash memory, static random access memory (SRAM), etc.), and a data storage device, which communicate with each other via a bus.

In alternative embodiments, a processor represents one or more general-purpose processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also be one or more special-purpose processing devices such as an application specific integrated circuit (ASIC), a field programmable gate array (FPGA), a digital signal processor (DSP), network processor, or the like. In alternative embodiments the processor is configured to execute the instructions (e.g., processing logic) for performing the operations and steps discussed herein.

In alternative embodiments a computer system used to practice the invention further comprises a network interface device. The computer system also may include a video display unit (e.g., a liquid crystal display (LCD) or a cathode ray tube (CRT)), an alphanumeric input device (e.g., a keyboard), a cursor control device (e.g., a mouse), and a signal generation device (e.g., a speaker).

In alternative embodiments, the data storage device (e.g., drive unit) comprises a computer-readable storage medium on which is stored one or more sets of instructions (e.g., software) embodying any one or more of the protocols, methodologies or functions of this invention. The instructions may also reside, completely or at least partially, within the main memory and/or within the processor during execution thereof by the computer system, the main memory and the processor also constituting machine-accessible storage media. The instructions may further be transmitted or received over a network via the network interface device.

In alternative embodiments the computer-readable storage medium is used to store data structure sets, e.g., for the capture, analyze, and display optical coherence tomography images of the neural tissue to monitor for a physiological event and/or a state prior to the physiological event. Data structure sets and patient or user profiles may also be stored in other sections of computer system, such as static memory.

In alternative embodiments, while the computer-readable storage medium in an exemplary embodiment is a single medium, the term “machine-accessible storage medium” can be taken to include a single medium or multiple media (e.g., a centralized or distributed database, and/or associated caches and servers) that store the one or more sets of instructions or data sets. In alternative embodiments the term “machine-accessible storage medium” can also be taken to include any medium that is capable of storing, encoding or carrying a set of instructions for execution by the machine and that cause the machine to perform any methods of the invention. In alternative embodiments the term “machine-accessible storage medium” can include a solid-state memory, or an optical or a magnetic media.

In alternative embodiments, information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

In alternative embodiments, modules, circuits, and algorithm steps needed to practice this invention may be implemented as electronic hardware, computer software, or combinations of both. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

In alternative embodiments, the invention provides computer systems comprising a processor and a data storage device wherein said data storage device has stored thereon: (a) a computer program product for implementing a computer-implemented method of the invention; (b) a computer program product for processing data of the invention; (c) a Graphical User Interface (GUI) computer program product of any of the invention; (d) a computer system of the invention; (e) a non-transitory memory medium of the invention; (f) a computer-readable storage medium of the invention; (g) a computer program product of the invention; (h) a computer program storage device of the invention; (i) a computer or equivalent electronic system of the invention; (j) a system of the invention; (k) a computer-implemented system of the invention; (1) a computer-implemented method for displaying feed data of the invention; (m) a storage device storing program instructions executable by a processor of the invention; or, (n) a combination thereof.

### Kits and Instructions

The invention provides kits comprising compositions and methods of the invention, including instructions for use thereof. In alternative embodiments, the invention provides kits comprising a composition or a product of manufacture of the invention; wherein optionally the kit further comprises instructions for practicing a method of the invention. In alternative embodiments, the invention provides portable and/or small kits that comprise an exemplary product of manufacture of the invention.

A number of embodiments of the invention have been described. Nevertheless, it can be understood that various modifications may be made without departing from the spirit and scope of the invention. Accordingly, other embodiments are within the scope of the following claims.

What is claimed is:

1. A method for using optical coherence tomography to monitor for a physiological event and/or a state prior to the physiological event in an individual, comprising:
(a) scanning a neural tissue with an optical coherence tomography (OCT) at a predetermined location, and generating an optical coherence signal;
(b) analyzing characteristics of the optical coherence signal on the neural tissue as a function of time; and
(c) determining OCT signal changes before the physiological event in the neural tissue becomes clinically manifested.

2. The method of claim 1, wherein:
(a) the method further comprises determining a signature signal that comprises changes, or a repeatable pattern, in signal intensity specifically associated with a seizure or a pre-seizure activity;
(b) the method is a computer implemented method and the method further comprises use of a computer program product to capture, analyze, and display optical coherence tomography images of the neural tissue to monitor for a physiological event and/or a state prior to the physiological event;
(c) the physiological event is a seizure;
(d) the physiological event is a seizure, and the method further comprises mediating or manipulating the neural activity of the neural tissue before onset of the seizure upon a determination of a signature temporal pattern;
(e) the physiological event is a seizure, and the method further comprises mediating or manipulating the neural activity of the neural tissue before onset of the seizure upon a determination of a signature temporal pattern, and the mediating of the neural activity of the neural tissue comprises a mediation sufficient to prevent the seizure;
(f) the method of (e), wherein the mediating of the neural activity of the neural tissue comprises a mediation sufficient to reduce the time (duration) of and/or the severity of seizure;
(g) determining the optical intensity of the optical coherence signal comprises determining a threshold value of the optical intensity;
(h) determining a threshold value of the optical intensity comprises determining a threshold value during one or more time windows of decreasing optical intensity;
(i) determining a signature signal of the optical intensity of the optical coherent signal comprises determining a threshold value of the time derivative of the optical intensity;
(j) determining a threshold value of the time derivative of the optical intensity comprises determining a threshold value of the time derivative of the optical intensity during one or more time windows of decreasing optical intensity;
(k) the optical coherence tomography is supplied through a fiber optic endoscope, or the optical coherence tomography is supplied through a fiber optic endoscope, and the method further comprises implanting an endoscope into a seizure foci of an individuals with epilepsy, wherein optionally the detecting of a pre-seizure state acts as an early warning device;
(l) the method further comprises utilizing variations of optical coherence tomography on the neural tissue with either broadband or specific wavelengths of radiation in the visible, near-infrared, and/or infrared region;
(m) the method further comprises determining a signature signal that comprises changes, or a repeatable pattern, in signal intensity associated with a seizure or a pre-seizure activity;
(n) the method further comprises determining a signature signal that comprises changes, or a repeatable pattern, in signal or optical intensity associated with pathologic brain swelling before measurable late sequelae of increased intracranial pressure or hemodynamic changes;
(o) the method, by detecting signal or optical intensity associated with pathologic brain swelling before measurable late sequelae of increased intracranial pressure or hemodynamic changes, acts as an early warning of increased intracranial pressure or hemodynamic changes;
(p) the method further comprises using an optical fiber edema probe incorporated into an intracranial monitoring device or inserted as a standalone probe into an area of interest in the neural tissue, and analyzing optical scattering and absorption related changes to provide an edema index on a continuous basis; or
(q) the method further comprises determining the OCT signal changes comprises use of light polarization information, the optical phase of the signal, spectroscopic information, and changes thereof.

3. A method for using optical parameters in detection of a seizure and/or a pre-seizure state in an individual, comprising:
(a) scanning a neural tissue with optical coherence tomography;
(b) detecting a magnitude of optical intensity reflected by the neural tissue as a function of time; and
(c) detecting changes in neural architecture that result from water and ion migration preceding and during specific brain electrical activity,
wherein detecting changes in the neural architecture detects a seizure and/or a pre-seizure state.

4. The method of claim 3, wherein:
(a) the method further comprises determining a signature signal that comprises changes, or a repeatable pattern, in optical intensity specifically associated with a seizure or a pre-seizure activity;
(b) the method is a computer implemented method and the method further comprises use of a computer program product to capture, analyze, and display optical coherence tomography images of the neural tissue;
(c) the optical coherence tomography is supplied through a fiber optic endoscope, or the optical coherence tomography is supplied through a fiber optic endoscope, and the method further comprises implanting an endoscope into a seizure foci of an individuals with epilepsy, wherein optionally the detecting of a pre-seizure state acts as an early warning device;
(d) the method further comprises utilizing variations of optical coherence tomography on the neural tissue with either broadband or specific wavelengths of radiation in the visible, near-infrared, and/or infrared region;
(e) the method further comprises determining a signature signal that comprises changes, or a repeatable pattern, in signal intensity associated with a seizure or a pre-seizure activity;
(f) the method further comprises determining a signature signal that comprises changes, or a repeatable pattern, in signal or optical intensity associated with pathologic brain swelling before measurable late sequelae of increased intracranial pressure or hemodynamic changes;
(g) the method, by detecting signal or optical intensity associated with pathologic brain swelling before measurable late sequelae of increased intracranial pressure or hemodynamic changes, acts as an early warning of increased intracranial pressure or hemodynamic changes;
(h) the method further comprises using an optical fiber edema probe incorporated into an intracranial monitoring device or inserted as a standalone probe into an area of interest in the neural tissue, and analyzing optical scattering and absorption related changes to provide an edema index on a continuous basis; or
(i) the method further comprises determining the OCT signal changes comprises use of light polarization information, the optical phase of the signal, spectroscopic information, and changes thereof.

5. A method for using optical parameters in detection of seizure and pre-seizure states comprising:
(a) scanning a neural tissue with an optical coherence tomography;
(b) detecting a magnitude of optical intensity reflected by neural tissue as a function of time; and
(c) detecting changes in cell volume and detecting changes in the extracellular space which decrease optical scattering through affected neural tissue,
wherein detecting changes in the cell volume and extracellular space which decrease optical scattering detects a seizure and/or a pre-seizure state.

6. The method of claim 5, wherein:
(a) the method further comprises determining a signature signal that comprises changes, or a repeatable pattern, in optical intensity specifically associated with a seizure or a pre-seizure activity;
(b) the method is a computer implemented method and the method further comprises use of a computer program product to capture, analyze, and display optical coherence tomography images of the cell or neural or extracellular tissue;
(c) the optical coherence tomography is supplied through a fiber optic endoscope, or the optical coherence tomography is supplied through a fiber optic endoscope, and the method further comprises implanting an endoscope into a seizure foci of an individuals with epilepsy, wherein optionally the detecting of a pre-seizure state acts as an early warning device;
(d) the method further comprises utilizing variations of optical coherence tomography on the neural tissue with either broadband or specific wavelengths of radiation in the visible, near-infrared, and/or infrared region;
(e) the method further comprises determining a signature signal that comprises changes, or a repeatable pattern, in signal intensity associated with a seizure or a pre-seizure activity;
(f) the method further comprises determining a signature signal that comprises changes, or a repeatable pattern, in signal or optical intensity associated with pathologic brain swelling before measurable late sequelae of increased intracranial pressure or hemodynamic changes;
(g) the method, by detecting signal or optical intensity associated with pathologic brain swelling before measurable late sequelae of increased intracranial pressure or hemodynamic changes, acts as an early warning of increased intracranial pressure or hemodynamic changes;
(h) the method further comprises using an optical fiber edema probe incorporated into an intracranial monitoring device or inserted as a standalone probe into an area of interest in the neural tissue, and analyzing optical scattering and absorption related changes to provide an edema index on a continuous basis; or
(i) the method further comprises determining the OCT signal changes comprises use of light polarization information, the optical phase of the signal, spectroscopic information, and changes thereof.

7. A product of manufacture or a device comprising an optical coherence tomography capable of monitoring for a physiological event and/or a state prior to the physiological event in an individual, comprising:
(a) scanning a neural tissue with an optical coherence tomography (OCT) at a predetermined location, and generating an optical coherence signal;
(b) analyzing characteristics of the optical coherence signal on the neural tissue as a function of time; and
(c) determining OCT signal changes before the physiological event in the neural tissue becomes clinically manifested.

8. The product of manufacture or device of claim 7, wherein:
(a) the product of manufacture or a device can determine a signature signal that comprises changes, or a repeatable pattern, in signal intensity specifically associated with a seizure or a pre-seizure activity, or
(b) wherein the product of manufacture or device further comprises a computer program product for enabling a computer implemented method to capture, analyze, and display optical coherence tomography images of the neural tissue to monitor for a physiological event and/or a state prior to the physiological event.

9. The product of manufacture or device of claim 7, further comprising:
(a) an artificial neural-network (ANN) based computer chip or a computer program product to enhance the specificity of an imaging system;
(b) a computer program product having a computer implemented method or a software programmed for screening for a “yes or no” response;
wherein optionally for a yes (optionally, yes for seizure or edema)) or a no (optionally, no for seizure or edema) basis,
and optionally comprising a memory, and a relaying or a telecommunication or an internet relay, and/or a display for a user component to store, transmit and/or display the “yes or no” response; or
(c) a sensor, wherein the sensor can optionally detect and/or record radiation exposure specific or cumulative for an operator and/or for subject safety.

10. A computer-implemented method for, or capable of, implementing a method of claim 1.

11. A computer program product for processing data acquired in implementing a method of claim 1.

12. A storage device storing program instructions executable by a processor to run, process and/or implement a computer-implemented method of claim 1.

13. A computer-implemented system for providing an application access to an external data source or an external server process via a connection server, and providing the ability to store values associated with the plurality of data points and/or the plurality of data elements, and an application for running, processing and/or implementing a computer-implemented method of claim 1.

14. A system, comprising: a memory configured to: store values associated with a plurality of data points and/or a plurality of data elements, and a processor adapted to execute program code stored in the memory to: run, process and/or implement a computer-implemented method of claim 1.

15. A computer program product for processing data acquired in implementing a computer-implemented method of claim 13.

16. A storage device storing program instructions executable by a processor to run, process and/or implement a computer-implemented method of claim 13.

17. A computer-implemented system for providing an application access to an external data source or an external server process via a connection server, and providing the ability to store values associated with the plurality of data points and/or the plurality of data elements, and an application for running, processing and/or implementing a computer-implemented method of claim 13.

18. A system, comprising: a memory configured to: store values associated with a plurality of data points and/or a plurality of data elements, and a processor adapted to execute program code stored in the memory to: run, process and/or implement a computer-implemented method of claim 13.

19. A computer program product for processing data acquired in implementing a method of claim 3.

20. A computer program product for processing data acquired in implementing a method of claim 5.
